(19) United States
(12) Patent Application Publication
(10) Pub. No.: US 2014/0274965 A1
(43) Pub. Date: Sep. 18, 2014

(54) Drink Product and Use Thereof

(71) Applicant: HYGIA PHARMACEUTICALS, LLC, Jupiter, FL (US)

(72) Inventor: Barbara Brooke Jennings, Palm Beach Gardens, FL (US)

(73) Assignee: HYGIA PHARMACEUTICALS, LLC, Jupiter, FL (US)

(21) Appl. No.: 13/803,716

(22) Filed: Mar. 14, 2013

(51) Int. Cl. A61K 31/6615

(52) U.S. Cl. CPC A61K 31/6615
USPC 514/102

(57) ABSTRACT

A drink product having as an active ingredient an inositol hexaphosphate and optionally additional inositol moieties. Hexaphosphates for use in the invention include myoinositol hexaphosphate and/or any of the other 8 optical isomers thereof. The optional additional inositols include myoinositol and/or any of the other 8 optical isomers thereof. Uses for prevention, treatment, and reduction in risk of developing or progression of a number of conditions are disclosed.

### FIELD OF THE INVENTION

This invention relates to certain drink products that generally are aqueous solutions containing myoinositol hexaphosphate (and or any of the optical isomers thereof) optionally enriched with any or all of myoinositol, optical isomers of myoinositol, electrolytes, flavors, vitamins, free radical scavengers, and sweeteners. The invention further relates to (a) the treatment of and/or (b) the prevention of and/or (c) the reduction in the risk of developing various conditions such as cancers, particularly, lung, skin, prostate, and colon cancers.

### BACKGROUND OF THE INVENTION

Sun tanning is the result of sun radiation and free radical damage. This occurs when radiation from the sun is converted into oxygen-free radicals (reactive oxygen species, aka ROS) that damage the skin and its most of its support structure upon long term exposure. Free radical scavengers as well as myoinositol hexaphoshosphate (IP6) have been shown to neutralize this oxidative stress in the skin which can be caused by both UVA and UVB radiation and prevent three types of skin cancers.

According to the CDC, cigarette smoking is the number one risk factor for lung cancer. There are few targeted treatment options. In the United States, cigarette smoking causes about 90% of lung cancers. The world's two most populous nations—India and China—are home to more smokers than the entire population of the European Union. In China, more than 300 million people are tobacco users, while India adds another 275 million to the tally.

A study by the George Institute of Global Health in 2010 revealed that the Asia-Pacific region is home to 30 percent of the world's smokers. The impact of smoking is greatest in these countries because smokers who develop lung cancer are diagnosed at later stages of the disease and receive less effective treatment. People who smoke are 15 to 30 times more likely to get lung cancer or die from lung cancer than people who do not smoke.

Existing literature has demonstrated a clear therapeutic benefit of inositol-1,2,3,4,5,6-hexakisphosphate, referred to herein as IP6, in high fiber diets and a growing number of scientific studies have demonstrated that IP6 has anti-proliferative and tumor suppressive properties and cancer prevention properties in numerous tumor types including skin cancers. The best human clinical evidence to date for cancer prevention in smokers using an oral pill form of administration is based on human clinical trials of smokers with dysplastic lung lesions that were reversed taking oral myoinositol.

Oral administration of IP6 has been shown to protect against lung and colon cancer and the prevention UVA/UVB induced skin tumors in basal, squamous, and melanoma skin tumors.

IP6 is a (poly) phosphorylated carbohydrate, ubiquitous in nature and synthesized in all animal cells. It functions as the principle storage form of phosphorous in many plants. It also occurs naturally in cereal grains, beans, brown rice, corn, sesame seeds, wheat bran, and other high fiber foods. Indeed, studies suggest that some of the health benefits afforded by eating a high fiber diet can be attributed to IP6 action. Copious amounts of high fiber is not practical for everyone and the human small intestine has very limited enzymes to break down this phosphate in order to reap the anti-cancer benefits associated with this compound. Furthermore, this phosphate gets bound to food proteins in the intestine and consequently largely degraded by stomach acids. Therefore a supplement for oral or an ionized aqueous administration having IP6 phytate is highly desirable.

Unlike rodents, the small intestine of humans express low levels of phytase, and this enzyme is required to metabolize IP6. Therefore. IP6 is not a source of dietary inositol and phosphate. Rather, myoinositol polyphosphates are synthesized within the body directly from phosphate and inositol, the latter being derived from glucose. IP6 ranges in concentration from 10 to 100 μM in mammalian cells. Importantly, phytic acid chelates several important minerals including zinc, iron, and to a lesser extent calcium and magnesium.

Inositol phosphates function as intracellular signaling intermediates that regulate a number of critical biochemical pathways in mammalian cells including cell cycle progression, cell differentiation, survival, migration, intracellular vesicle transport, metabolism and autophagy. Indeed, a key regulator of the activity of phosphatidylinositol (PtdIns) lipid substrates is the enzyme phosphatidylinositol 3-kinase (PI3K) which is the major cancer causing protein involved in all cancerous tumors. This enzyme phosphorylates the 3-OH residue of the myo-inositol ring of three specific PtdIns; phosphatidylinositol, phosphatidylinositol-4-phosphate, and phosphatidylinositol-4,5-bisphosphate. PI3K activity plays an important role in regulating the activity of other kinases that play important roles in metabolism and cell growth and survival including AKT (protein kinase B) and mTOR (mammalian target of rapamycin). Indeed, dysfunction and/or aberrant regulation of the PI3K pathway is implicated in cancers of the colon, breast, brain, liver, stomach, and lung. Therefore, development of potent and selective PI3K inhibitors as novel cancer therapeutics is currently the focus of intensive research efforts. However, first generation PI3K inhibitors developed in the art exhibited toxic side effects and poor pharmacological properties and selectivity, and as such were only effective in preclinical models. Alternatively, there exists a large amount of epidemiological data indicating that inositols are protective against certain forms of cancer. Indeed, a growing body of literature has linked the therapeutic benefits to natural products, such as IP6. Although IP6 has been shown to function as a free radical scavenger, other studies have demonstrated that it also functions as a PI3K inhibitor. Furthermore, several studies have shown that the ability of IP6 to block cell transformation or growth of tumor cells is directly related to inhibition of PI3K activity. For example, Huang et al. (1997) reported that IP6 at a concentration range of 0.5-2 mM showed a dose-dependent inhibition of EGF or TPA-induced cell transformation in JB6 cells as measured by three-dimensional growth in soft agar. This activity was linked to direct inhibition of PI3K. Similarly, Gu et al. (2009) showed that IP6-mediated growth inhibition of the prostatic adenocarinoma cell line PC-3 was correlated with reduced phosphorylation levels of the p85 subunit of PI3K at Tyr⁴⁵⁸ and phosphoinositide-dependent kinase-1 PDK1) at Ser²⁴¹. IP6 also caused a strong decrease in levels of integrin linked kinase 1 (ILK1) and cyclin DI and reduced levels of phosphorylated AKT and GSK3α/β in these cells.

Based on these results and other data in both experimental animal models and human clinical trials demonstrating a therapeutic benefit of IP6 in preventing cancer, these products can be used as a prophylactic to protect against environmental insults that are implicated in tumor promotion via modulation of the PI3K/AKT/mTOR pathway.

More people die of lung cancer in the U.S. than any other type of cancer. This is true for both men and women. Most importantly, the overall 5-year survival rate for patients diagnosed with lung cancer is ˜12% for men and ˜16% for women. While smoking is the main cause of lung cancer, e.g. male and female smokers are 23 and 13 times more likely to develop lung cancer than non-smokers, respectively, 15% of all lung cancer patients have never smoked. Most importantly, no reliable metrics exists that can predict whether a smoker will develop lung cancer. Since most lung cancer patients are diagnosed with advanced disease, this knowledge is critical for developing adjuvant and front line therapies to improve survival of lung cancer patients.

Several studies have shown that myoinositol has protective effects in experimental models of lung cancer. For example, Estensen and Wattenberg reported that a diet containing 3% myo-inositol fed beginning 1 week after exposure to benzo[a]pyrene reduced the number of pulmonary adenomas by 40%.

These studies are significant in that benzo[a]pyrene is a polycyclic aromatic hydrocarbon found in automobile exhaust fumes, cigarette smoke, and charbroiled food metabolites, and its metabolites are both highly mutagenic and carcionogenic. Indeed, benzo[a]pyrene has been directly linked to lung and colon cancer. These results were confirmed by Hecht et al. and Witschi et al using a similar model of benzo[a]pyrene with or without 4-(methylnitrosamino)-1-(3-pyridyly)-1-butanone, the latter of which is also a component of cigarette smoke. Moreover, Witschi et al further demonstrated that a diet supplemented with 10 g per kilogram of myoinositol and 0.5 mg/kg dexamethasone (a steroid) produced a significant reduction in both lung tumor multiplicity and in tumor incidence as compared to a control diet in mice exposed to tobacco smoke. In these studies, mice were exposed to 71 mg total suspended particulates (TSP)/m³ for the first two weeks, 86 mg/m³ for three additional weeks, and 132 mg/m³ for an additional 17 weeks for 6 hr per day, 5 days per week. In these studies, the diet containing myo-inositol/dexamethasone proved to be a highly effective chemopreventive regimen. The number of tumors per lung in animals fed myoinsoitol+dexamethasone and exposed to tobacco smoke actually approached the tumor multiplicity in animals exposed to filtered air.

Recently, a landmark study published by Gustafson et al. in the high impact journal Science Translational Medicine revealed that the PI3K pathway is activated in cytologically normal proximal airway epithelial cells in smokers with lung cancer and with dysplastic lesions. In these studies, PI3K activity was measured biochemically in airway biopsies by Western blot analysis and computationally by genome-wide micro-array analysis. This analysis revealed that the PI3K pathway was significantly up regulated in the airways of smokers with lung cancer as compared to controls, and genes that play a role in phosphatidylinositol signaling pathway were also significantly up regulated. Methodologies used to evaluate lung cancer patients were confirmed by demonstrating that PI3K signaling was also up regulated in basal-like and HER2-overexpressing breast tumors that lack PTEN function, a negative regulator of the PI3K pathway. Moreover, this analysis showed that the PI3K pathway signature was expressed at higher levels in lung tumors as compared to adjacent normal tissue, indicating that PI3K activation is important for lung cancer tumorigenesis.

The work by Gustafson et al. demonstrated that smokers with regression of dysplasia following treatment with myo-inositol showed significant (p<0.05) increased expression of genes that are repressed on PI3K activation in vitro, while smokers who did not respond to myo-inositol treatment had no change in the PI3K gene sets. The decrease in PI3K activity in patients who respond to myoinositol suggests that regression of dysplasia is associated with a reduction of PI3K pathway activity in the proximal airway. The authors subsequently showed that treatment of a lung adenocarcinoma cell line with myoinositol resulted in dose-dependent decreases in PI3K activity. Indeed, the median effective concentration (EC₅₀) of myoinositol for PI3K inhibition was 7.3×10⁻⁸M. This EC₅₀ is comparable to the myoinositol doses given for regression of dysplastic lesions to high-risk smokers, which was 9 g twice daily (Lam S et al., 2006). These findings suggest that the cancer chemoprevention properties of myoinositol are most likely related to inhibition of PI3K activity.

Safety and efficacy of myoinositol has already been established in a Phase I clinical trial-Mayo Clinic (Lam et al. 2006). In this trial, sixteen smokers between 40 and 70 years of age with greater or equal to 30 pack-years of smoking history and one or more sites of bronchial dysplasia were enrolled in a dose escalation study of myo-inositol for one month. Subsequently, ten subjects were enrolled in a 3 month study employing the maximum tolerated dose, which was found to be 18 grams per day administered via oral administration. Side effects, when present, were mild and mainly gastrointestinal in nature. More importantly, a significant increase in the rate of regression of preexisting dysplastic lesions was observed (91% versus 48%; P=0.014) and a statistically significant reduction in the systolic and diastolic blood pressures by an average of 10 mm Hg was observed. Therefore, these data demonstrate that myoInositol in a daily dose of 18 grams per day for 3 months is safe and well tolerated and produces significant regression of pre-existing dysplastic lesions in smokers.

A wealth of epidemiological data exists showing that high-fiber diets are associated with lower risks of large intestinal cancer. Since IP6 levels are high in nuts and grains, several studies have directly evaluated the cancer fighting properties of IP6 in experimental animals of colon cancer. For example, Shamsuddin et al. (1988) first demonstrated that rats r supplemented with 1% sodium IP6 before or two weeks post treatment with the carcinogen azoxymethane exhibited a 35% decrease in large intestinal cancer. A follow up study conducted by Shamsuddin and Ullah (1989) further showed that treatment of rats with IP6 was effective in significantly (p=0.02) reducing large intestinal cancers even when the treatment was begun 5 months after carcinogenic induction with azoxymethane. Consistent with these data, IP6 administration also reduced early biomarkers of colon cancer risk including the degree of aberrant crypt loci and labeling index of crypt cells in rodents (Jenab and Thompson, 1998; Norazalina et al., 2010). These studies are consistent with other reports indicating that IP6 inhibits growth of the human colon cancer cell lines in vitro (Sakamoto et al. 1993; Tain and Song, 2006), lowered their metabolic activity (Schroterova et al., 2010), and decreased the number of cells in S-phase by inducing G0/G1 arrest (El-Sherbiny et al. 2001).

TABLE A
Antitumor effect of inositol hexaphosphate (IP6) in-vitro

| Organ/Tissue | Species | Cell Line | Investigator |
|---|---|---|---|
| Blood | Human | Erythroleukemia K562 cell | Shamsuddin et al |
| | | line, K562 1 human bone | Deliliers et al. ( |
| | | marrow | |
| Colon | Human | Adenocarcinoma, | Sa Yang & Shamsuddin |
| | | HT-29 cell line | Samoto et al |
| Lung | Rat | Trachael epithelial + | Arnold et al. |
| | | B[a]P | |
| Liver | Human | Hep G2 cells | Vucenik et al. |
| Mammary | Human | Adenocarcinoma | Shamsuddin et al. |
| | | MCF-7, MDA-MB 231 | |
| | | cells | |
| Uterine cervix | Human | HeLa cells | Ferry et al. |
| Prostate | Human | Adenocarcinoma | Shamsuddin & Yang Zi |
| | Human | P DU145 cells DC-3 cell line | et al. |
| | | | Singh et al. |
| Skin | Mouse | JB6 cells | Huang et al |
| | Mouse | HEL-30 cells | Nickel & Belury |
| | mice | melanoma line HTB68 | Babich et al. |
| | | also see references below for | Rizvi, et al |
| | | other skin cancer cell lines and | |
| | | investigator authors | |
| Soft tissue | Mouse | 3T3 fibroblast | Babich et al. Vucenik et |
| | Human | Rhabdomyosarcoma, RD cells | al. |

Inositols are available as dietary supplements. IP6, inositol, and D-chiroinositol are sold in pill or powder form. Inositol and D-chiroinositol are advertised as members of the vitamin B complex (Vitamin B₈). However, this classification is outdated since they are produced from glucose in the body and as such are not essential nutrients. Inositol is advertised as useful in ameliorating symptoms associated with anxiety and depression. In double blinded clinical trials, D-chiro-inositol provided a substantial benefit to women with polycystic ovary syndrome (Nestler, 1999; Iuorno, 2002).

### OBJECTS OF THE INVENTION

It is an object of the invention to provide a liquid and/or ionized aqueous product containing myoinositol hexaphosphate (and/or optical isomer thereof) as a dietary supplement and/or medical treatment.

It is another object of the invention to provide a liquid product containing myoinositol hexaphosphate (and/or optical isomer thereof) together with myo-inositol (and/or optical isomer thereof) as a dietary supplement and/or as a medical treatment.

A still further object of the invention is to provide a liquid product as described in any of the foregoing objects of the invention which further contain a free radical scavenger.

Yet another object of the invention is to provide a liquid product as described in any of the foregoing objects of the invention further comprising at least one non-inositol B vitamin.

An even further object of the invention is to provide a liquid product as described in any of the foregoing objects of the invention further comprising at least one additional nutritional supplement or neutraceutical component.

A still further object of the invention is the use of any of the foregoing liquid products as a nutritional supplement in humans or animals.

Yet another object of the invention is the use of any of the foregoing liquid products in an oral method for the treatment, prevention, and/or reduction of risk of a cancer selected from the group consisting of skin (including squamous cell carcinoma, basal cell carcinoma, and melanoma), lung, pancreatic, breast, liver, blood, soft tissue, ovarian, prostate and/or colon cancers (see table and references), or in a method of the treatment of damage from or prevention of damage from or reduction of the risk of damage from reactive oxygen species in a human or animal in need thereof.

Still another object of the invention is the use of any of the foregoing liquid products in an oral neutraceutical or nutritional supplement product, where such product is used in a method for the treatment, prevention, and/or reduction of risk of a cancer selected from the group consisting of skin, lung, prostate, and/or colon cancers, or in a method of the treatment of damage from or prevention of damage from or reduction of the risk of damage from reactive oxygen species in a human or animal in need thereof or as a nutritional supplement for use in the support of health with respect to any of the above.

Still other objects of the invention will be apparent to those of ordinary skill once having the benefit of the present disclosure.

### DETAILED DESCRIPTION OF THE INVENTION

The present invention is an oral, aqueous liquid formulation containing myoinsoitol hexaphosphate (IP6) in an effective amount as a nutritional supplement; and/or an effective amount as a medicinal for the treatment of, prevention of, or reduction of the risk of developing a cancer of the skin, lung, prostate, or colon; and/or an effective amount for the treatment of, prevention of, and/or reduction in the risk of damage to cells caused by reactive oxygen species (ROS). Other utilities and aspects of the present invention include protection of those going to high elevations (particularly pilots, extreme mountain climbers, as well as astronauts) as a protectant prophylactically against radiation exposure due to lesser protective effects of the atmosphere against ambient radiation, and environmental toxins, i.e car exhaust, pollution, radiation, and known toxic free radical generating chemicals. (One particularly troublesome group of compound in the environment includes phthalates, which are known to generate free radical oxygen species in vivo, and thus, the present invention is useful to offset the undesirable effects thereof.) Similarly, the present invention is also useful as a prophylactic measure against some of the accidental or routine radiation exposure involved with nuclear power plant operators and those involved in mining and refining of radioactive materials. It should be realized that the present invention is not expected to fully cure or fully prevent such effects of radiation exposure, but is of use in minimizing or reducing the effects that would result in the absence of use of the invention or any other preventative measure. Still further utilities for the present invention include usage before, during, or after radiation treatments, which radiation treatments are either for disease treatment (as in radiation for cancer therapy) or diagnostic purposes, such as CAT scans, X-rays, etc., as a partial protectant against at least some of the undesirable effects of such radiation exposure.

In addition to the IP6 (and/or optical isomer thereof) and water, the formulations optionally may also contain, one or more of myoinositol (and/or optical isomer thereof). Still further optional components of the present formulation include nutritionally acceptable monovalent electrolytes (including without being limited thereto, cations such as, without limitation sodium, and potassium, and anions such as, without limitation chloride; non-limiting examples of which include sodium chloride, and potassium chloride) as are known in the nutritional supplement arts and/or vitamins (including, without limitation thereto the B vitamins (including without limitation thiamine (vitamin B1); riboflavin (vitamin B2); niacin and/or nicotinamide (vitamin B3); pantothenic acid (vitamin B5); pyridoxine, pyridoxal, pyridoxamine, and/or pyridoxine HCl (vitamin B6); biotin (vitamin B7); folic acid (vitamin B9), and cobalamine (vitamin B12)) and vitamin C), as well as herbal extracts. (Nutritionally available salts and esters of the various vitamins may be used in place of the specifically stated vitamin.) The above vitamins and electrolytes are used in amounts such that from 1 to 4 doses (whether as 30 ml, 60 mil, 90 ml, 120 ml, 150 ml, 180 ml, or 240 ml per dose) delivers from ¼ to the full US RDA independently for each one present in a particular formulation (although lesser amounts are acceptable but just not preferred). The formulations of the invention may also optionally contain one or more of flavors (such as without limitation, coconut, grape, blueberry, pomegranate, apple, strawberry, kiwi, dragonfruit, lemon, lime, raspberry, mango, etc), sweeteners (such as without limitation, sucrose, fructose, glucose, stevia, aspartame, rebaudioside A, sucralose, mannitol, xylitol, syrups, etc.), colorings, stabilizers, pH adjusters, preservatives (such as without limitation, sodium benzoate), etc, as well as thickeners and processing aids as may be generally known in the beverage and/or liquid nutritional supplement arts in amounts generally known as acceptable in the art.

Effective amounts of the IP6 component (or optical isomers thereof, preferably myoinositol hexaphosphate), when used without other inositol components range from about 0.5 to about 4% w/v, preferably about 0.75 to about 3% w/v, more preferably about 1 to about 2.5% w/v, still more preferably about 1.1 to about 2% w/v, even more preferably 1.2 to about 1.5% w/v, and most preferably about 1.25% w/v, with a daily dosing typically in the range such that about 0.5 to about 8 grams per day, preferably about 1.0 to about 7 grams per day, more preferably about 1.2 to about 6 grams per day, still more preferably about 2.4 to about 5.5 grams per day, yet more preferably about 3.6 to about 5.0 grams per day, and most preferably about 4.8 grams per day of myoinositol hexaphosphate (and/or an optical isomer thereof) is administered. When combined with myoinositol (and/or an optical isomer thereof), the hexaphosphates (preferably myoinositol hexaphosphate) are combined with the unphosphorylated inositols (preferably myoinositol, aka vitamin B11) in a ratio of the hexaphosphates (most preferably myoinositol hexaphosphate) to unphosphorylated inositols (most preferably myoinositol) of about 6:1 to 1:6, preferably about 5:1 to about 1:5, more preferably about 4:1 to about 1:4, still more preferably about 3:1 to about 1:3, even more preferably about 2:1 to about 1:2, most preferably about 1:1, each being a molar ratio. Other suitable ratios include, without limitation, preferably about 4.5:1 to about 1:1, more preferably about 4.25:1 to about 3:1, still more preferably about 4:1 of the hexaphosphates (most preferably myoinositol hexaphosphate) to unphosphorylated inositols (most preferably myoinositol) on a molar basis. Nonetheless, the specific concentration in a given drink may be more or less (including multiple times or fractions thereof (if the volume of the drink per daily dose is properly adjusted to compensate therefore). Thus, for example, for a 1.25% w/v solution of IP6, where a drink is intended to deliver 3000 mg/240 ml with a serving or dose size of 240 ml (i.e. to deliver a 6 g dose of myoinositol hexaphosphate in two 240 ml doses, typically administered as 240 ml twice daily), an alternate drink having 1500 mg/120 ml with a serving or dose size of 120 ml (either taken 4 times a day or in the situation where a lower dosing is desired, less frequently), or another alternate of 62.5 mg/5 ml where significantly lower amounts are desired. Adjustment of dosings for smaller than average or larger than average adults or for adolescents or children will generally be within the skill of one of ordinary skill in the art given the foregoing ranges for a typical adult. When used in combination with other inositol species, the specific amount of IP6 may be reduced somewhat, but preferably, the other inositol species are added to the above mentioned amounts of IP6. The optional inositol species other than IP6 are generally available in the art from various sources, but preferably, the invention has IP6 optionally with or without myoinositol as the only inositol species. When an IP6 is used in combination with an unphosphorylated inositol, the most preferred ratio is stated above is a molar ratio of 1:1 and in a most preferable, but non-limiting embodiment, the total daily dose of such a ratio is about 4.8 grams of the myoIP6 (or isomer thereof) together with 1.32 grams of the unphosphorylated myoinositol (or isomer thereof).

As to the isomers of myoinositol, there may be used scylloinositol, mucoinositol, D-chiroinositol, L-chiroinositol, neoinositol, alloinositol, epiinositol, and/or cisinositol, with myoinositol, and D-chiroinositol being preferred and myoinositol being most preferred. As to the isomers of the myoinositol hexaphosphate component, one may use the hexaphosphates of the corresponding myoinositol isomers set forth in the preceding sentence, with myoinositol hexaphosphate and D-chiroinositol hexaphosphate being preferred and myoinositol hexaphosphate being most preferred.

As the optional free radical scavengers, any orally acceptable free radical scavenger known in the art is a is acceptable for use in the present invention, for example, without limitation, N-Acetyl-L-cysteine, L-Ascorbic acid, Balsalazide (typically available as the disodium salt hydrate), Caffeic acid, (−)-Catechin gallate, Chlorogenic acid, Delphinidin chloride, Diosmin, Ellagic acid, (−)-Epicatechin, Fucoxanthin carotenoid antioxidant, (−)-Gallocatechin, (−)-Gallocatechin gallate, Ginkgolide B, 3-Hydroxytyrosol, Luteolin, Lycopene, Neochlorogenic acid, Resveratrol, Rutin hydrate, Seleno-L-methionine, Se-(Methyl)selenocysteine hydrochloride, and Sodium selenite, most preferably L-ascorbic acid. The optional orally acceptable free radical scavengers can be used in amounts generally known as acceptable in the art; when ascorbic acid is used, it is preferably present in amounts of about 30 mg/120 ml of solution (i.e., about 0.025% w/v) or 60 mg/120 ml of solution (about 0.05%0 w/v).

The present invention offers IP6 in an aqueous oral form as a means to reap the therapeutic benefits of this natural product, particularly in lieu of or in addition to a diet high in fiber. One of the advantages of the present invention is that the delivery of undigested phytate IP6 and the other inositol species are in a form in which they are not captured and broken down as readily as solid forms containing these agents.

As previously described, the present invention formulation is used for an oral delivery of the active agents therein for the prevention and/or treatment and/or reduction of the risk of developing cancers such as, without limitation, one or more of skin cancer (inclusive of squamous, basal, and melanoma), breast, prostate, ovarian, pancreatic, lung cancer, colon cancer liver cancer, bone cancer, soft tissue cancer (such as without limitation, muscular cancers) or a blood cancer (or as described in Table A above) and/or treatment of damage and/or prevention of damage and/or reduction of the risk of damage to cells by reactive oxygen species free radicals that are generated in the body due to environment or metabolic insult. Environmental or metabolic insult may arise from exposure to a wide variety of factors, including radiation, known carcinogens, and known free oxygen radical generating substances (such as in, without limitation, cigarette smoke, pollutants, radiation exposure (whether natural, or created in the workplace, or incident to diagnostic tests and medical treatments, etc.). For the above purposes, a suitable (non-limiting, exemplary) dose of a formulation of the present invention having about 1% to about 4% w/v of myoinositol hexaphosphate (and/or optical isomer thereof) therein in an amount of about 30 ml to about 480 ml, preferably about 60 ml to about 360 ml, more preferably 90 ml to about 270 ml, still more preferably 360 ml, about 120 ml to about 240 ml, most preferably about 180 ml to about 240 ml at least once to 2 times per day, with variations thereon that will be recognized by those of ordinary skill in the art for delivery of about 3 g to about 9 g of myoinositol hexaphosphate (and/or optical isomers thereof) on a daily basis for an average adult in 1-2, or 2-4, divided doses a day.

The formulations of the present invention most preferably have the inositol components thereof in solution and preferably are clear, although non-inositol portions of the formulation may make the solution less than completely clear without departing from the invention.

The following non-limiting examples are exemplary only and do not have any limiting effect on the present invention.

### Example 1

The following Table I presents a set of inositol hexaphosphate isomers (alone or with an unphosphorylated inositol) for use in the present invention. Table II specifies amounts of the hexaphosphated inositol and the unphosphorylated inositol components, and each is applied independently to the 90 combinations in Table I. For clarity, all of the “A” cells in Table I have the respective hexaphosphate mentioned in the left band column with no unphosphorylated inositol component. All of the “B” cells of Table I have the respective hexaphosphate in combination with unphosphorylated myo-inositol.

TABLE I

| Hexaphosphate | Unphosphorylated Component | | | | | | | | | |
|---|---|---|---|---|---|---|---|---|---|---|
| Component | A | B | C | D | E | F | G | H | I | J |
| 1. Myo- | None | Myo- | Scyllo- | Muco- | D-chiro- | L-chiro- | Neo- | Allo- | Epi- | Cis- |
| inositol | | inositol | inositol | inositol | inositol | inositol | inositol | inositol | inositol | inositol |
| Hexaphosphate | | | | | | | | | | |
| 2. scyllo- | None | Myo- | Scyllo- | Muco- | D-chiro- | L-chiro- | Neo- | Allo- | Epi- | Cis- |
| inositol, | | inositol | inositol | inositol | inositol | inositol | inositol | inositol | inositol | inositol |
| Hexaphosphate | | | | | | | | | | |
| 3. Muco- | None | Myo- | Scyllo- | Muco- | D-chiro- | L-chiro- | Neo- | Allo- | Epi- | Cis- |
| inositol | | inositol | inositol | inositol | inositol | inositol | inositol | inositol | inositol | inositol |
| Hexaphosphate | | | | | | | | | | |
| 4. D-chiro- | None | Myo- | Scyllo- | Muco- | D-chiro- | L-chiro- | Neo- | Allo- | Epi- | Cis- |
| inositol | | inositol | inositol | inositol | inositol | inositol | inositol | inositol | inositol | inositol |
| Hexaphosphate | | | | | | | | | | |
| 5. L-chiro- | None | Myo- | Scyllo- | Muco- | D-chiro- | L-chiro- | Neo- | Allo- | Epi- | Cis- |
| inositol | | inositol | inositol | inositol | inositol | inositol | inositol | inositol | inositol | inositol |
| Hexaphosphate | | | | | | | | | | |
| 6. Neo- | None | Myo- | Scyllo- | Muco- | D-chiro- | L-chiro- | Neo- | Allo- | Epi- | Cis- |
| inositol, | | inositol | inositol | inositol | inositol | inositol | inositol | inositol | inositol | inositol |
| Hexaphosphate | | | | | | | | | | |
| 7. allo- | None | Myo- | Scyllo- | Muco- | D-chiro- | L-chiro- | Neo- | Allo- | Epi- | Cis- |
| inositol | | inositol | inositol | inositol | inositol | inositol | inositol | inositol | inositol | inositol |
| Hexaphosphate | | | | | | | | | | |
| 8. epi- | None | Myo- | Scyllo- | Muco- | D-chiro- | L-chiro- | Neo- | Allo- | Epi- | Cis- |
| inositol | | inositol | inositol | inositol | inositol | inositol | inositol | inositol | inositol | inositol |
| Hexaphosphate | | | | | | | | | | |
| 9. Cis- | None | Myo- | Scyllo- | Muco- | D-chiro- | L-chiro- | Neo- | Allo- | Epi- | Cis- |
| inositol | | inositol | inositol | inositol | inositol | inositol | inositol | inositol | inositol | inositol |
| Hexaphosphate | | | | | | | | | | |

TABLE II
% are w/v unless noted otherwise

| | I | II | III | IV | V | VI | VII | VIII | IX |
|---|---|---|---|---|---|---|---|---|---|
| Hexaphosphated | 1% | 2% | 4% | 1% | 2% | 4% | 1% | 2% | 4% |
| inositol of Table 1 | | | | | | | | | |
| Unphosphorylated | 0.135% | 0.27% | 0.54% | 0.27% | 0.54% | 1.08% | 0.54% | 1.08% | 2.16% |
| inositol of Table 1 | | | | | | | | | |
| When present* | | | | | | | | | |
| Ascorbic acid | 0.025% | 0.025% | 0.025% | 0.05% | 0.05% | 0.05% | 0.1% | 0.1% | 0.1% |
| Water | qs to | qs to | qs to | qs to | qs to | qs to | qs to | qs to | qs to |
| | 240 ml. | 240 ml. | 240 ml. | 240 ml. | 240 ml. | 240 ml. | 240 ml. | 240 ml. | 240 ml. |

*Molar ratios of hexaphosphates to unphosphorylated inositol in above Table II are either 0.5:1 or 1:1, or 2:1

Each of the 810 formulations resulting from Tables I and II are packaged for introduction into the marketplace. For the sake of clarity, a Table I cell 1B Table II Col I formulation has 1% myoinositol hexaphosphate; 0.135% myoinositol.

### Example 2

The following Table III utilizes each of the separate formulations of Example I except that additional nutritional supplement components are added. The amounts in Table III are expressed as % of the US Recommended Daily Allowance (USRDA) for the particular component in question, or if in mg, then mg/240 ml of solution.

TABLE IIIA

| Vitamin Or | | | | | | | | | |
|---|---|---|---|---|---|---|---|---|---|
| electrolyte | a | b | c | d | e | f | g | h | i |
| B1 | 5% | 10% | 15% | 20% | 5% | 10% | 15% | 20% | 5% |
| B2 | 5% | 10% | 15% | 20% | 5% | 10% | 15% | 20% | 5% |
| B3 | 5% | 10% | 15% | 20% | 5% | 10% | 15% | 20% | 5% |
| B5 | 5% | 10% | 15% | 20% | 5% | 10% | 15% | 20% | 5% |
| B6 | 5% | 10% | 15% | 20% | 5% | 10% | 15% | 20% | 5% |
| B7 | 5% | 10% | 15% | 20% | 5% | 10% | 15% | 20% | 5% |
| B9 | 5% | 10% | 15% | 20% | 5% | 10% | 15% | 20% | 5% |
| B12 | 5% | 10% | 15% | 20% | 5% | 10% | 15% | 20% | 5% |
| sodium | 110 mg | 110 mg | 110 mg | 110 mg | 110 mg | 110 mg | 110 mg | 110 mg | 110 mg |
| potassium | 450 mg | 450 mg | 450 mg | 450 mg | 450 mg | 450 mg | 450 mg | 450 mg | 450 mg |

Each of the formulations in Tables 1-1 IA have the further optional electrolytes and supplemental components such as preservatives and stabilizers added in accordance with Table IIIB. Each of the resulting solution is bottled for distribution.

TABLE IIIB

| Component | i | ii | iii | iv | v | vi | vii | viii | ix |
|---|---|---|---|---|---|---|---|---|---|
| sodium | none | 55 mg | 110 mg | 220 | none | 55 mg | 110 mg | 220 | 110 mg |
| potassium | none | none | none | none | 450 mg | 450 mg | 450 mg | 450 mg | 450 mg |
| Sodium benzoate, | None | 0.1% | 0.1% | 0.1% | 0.1% | 0.1% | 0.1% | 0.1% | 0.1% |
| malic acid | none | 250 mg | 250 mg | 250 mg | 500 mg | 500 mg | 500 mg | 500 mg | 1000 mg |

### Example 3

A single specific formulation of the invention comprises:

distilled water

1:1 molar ratio Myo-Inositol hexaphosphate (myo-IP6) (4.8 grams) and myo-inositol (1.32 grams),

crystalline fructose,

citric acid (preservative),

vegetable juice (color),

natural flavor,

ascorbic acid (vitamin C),

sodium citrate (electrolyte/antioxidant),

monopotassium phosphate

niacin (B3),

pantothenic acid (B5),

pyridoxine hydrochloride (B6),

cyanocobalamine (B12)

The above components are dissolved in the water and bottled for distribution.

### Example 4

A particular formulation of the invention comprises

Carbonated water-Carbon dioxide,

Inositol hexaphosphate (4.8 grams) and myo inositol 1.32 grams (1:1 molar ratio),

Erythritol(Rebiana™),

citric acid (preservative),

vegetable juice (color),

natural flavor,

artificial flavors,

ascorbic acid (vitamin C),

sodium citrate (elecrolyte),

Monopotassium Phosphate

niacin (B3),

pantothenic acid (B5),

pyridoxine hydrochloride (B6),

cyanocobalamine (B12)

The above components are dissolved in the water and bottled for distribution.

### Example 5

A particular formulation of the invention comprises

Distilled and/or sterilized community tap water.

Myo inositol (4.8 grams)

granulated table sugar (14 grams),

citric acid,

high fructose corn syrup,

colors,

glucose,

fructose,

sodium citrate (preservative),

vegetable juice (color),

natural flavor,

ascorbic acid (vitamin C),

natural flavor,

sodium citrate (electrolyte),

monopotassium phosphate (electrolyte),

niacin (B3),

pantothenic acid (B5),

pyridoxine hydrochloride (B6),

cyanocobalamine (BI 2)

sufficient sodium chloride to bring the sodium content up to 110 mg/240 ml,

The above components are dissolved in the water and bottled for distribution.

### Example 6

800 mg of myoinositol hexaphosphate and 220 mg of myoinositol were dissolved in 240 ml of distilled water to form a drink product of the present invention. The drink product was consumed twice a day.

### Example 7

### Bottled Water of the Invention

Water, if not already either purified, distilled, or filtered is subjected to an operation to obtain the water in a purified, distilled, or filtered state, and if need be stored for future use. The water is then used to dissolve the components of one of the foregoing formulations, and the result is bottled into appropriate containers.

### Example 8

### Bottled Carbonated Soft Drink of the Invention

Purified, distilled, or filtered water as obtained in the initial step of Example 7 is used placed in a mixing tank. The various components of the soft drink, which typically do not include the inositol components of the present invention, typically sweeteners, “soft drink concentrate”, and flavor are added to the mixing tank. The inositol and hexaphosphate inositol components, either as dry powders or concentrate solutions in water or as a pre-blend is added to the mixing tank in one embodiment before “soft drink components” or thereafter in a second embodiment. The solution is then subjected to a carbonation process and the result is bottled in suitable containers.

### Example 9

### Bottled Ready to Drink Tea of the Invention

A typical available ready to drink tea of the invention is prepared by preparing such tea in the ordinary fashion for such tea and subjecting the tea to a pasteurization process. The components of the present invention other than the tea are added thereto (as powders) under asceptic bottling conditions or alternatively the components of the present invention other than the tea are dissolved in purified, distilled, or filtered water at high concentrations, subjected to asceptic filtration, and the asceptically filtered solution is added to the pasteurized tea under asceptic conditions.

### Example 10

### Bottled Juice Drink of the Invention

A bottled fruit juice of the invention can be prepared by merely taking an existing fruit juice drink after it has been pasteurized and fortifying it under asceptic conditions with the inositol hexaphosphate or a mixture of inositol hexaphosphate and inositol and continue to bottle it in appropriate containers under aseptic conditions.

### Example 11

### Inositol Hexaphosphate and Inositol Enriched Soft and Hard Drinks

Myo-inositol hexaphosphate and myo-inositol in a 1:1 molar ratio are added to known bottled soft (including bottled water) and/or hard drinks, including those marketed under various trade names including those products made or distributed under various Coco-Cola™ brands, Aquapure, Aquarius, Bacardi Mixers, Bacardi Premium Mixers, Barq's, Barrilitos, Beverly, Bright And Early, caffeine free Barq's, caffeine free Coca-cola, caffeine free Coke light/Diet Coke, Campbells, Cascal, cherry Coke, Chippewa, Citra, Coca-cola, Coca-Cola Zero, Cumberland Gap, DANNON *, DASANI, Delaware Punch, diet_Barq's, Diet cherry Coke, Diet Coke/Coca-Cola light, Diet Coke/Coca-Cola light with Lime, diet Fanta, diet Inca Kola, diet Mello Yello/Mello Yello Zero, Diet NESTEA*, diet Vanilla Coke, Dr Pepper, Evian, Fanta, Five Alive, Flavor Rage, Fresca, Fruitopia, FUZE, Georgia, glacéau smart water, glacéau vitamin water, glacéau vitamin water zero, Gold Peak, H2OK, Hi-C, Honest, Illy *, Inca Kola, Java Monster, Juan Valdez, Krest, Lift, Master Chill, Master Pour, McCafe, Mello Yello, Mezzo Mix, Minute Maid, Minute Maid Enhanced, Minute Maid Juices To Go, Minute Maid Soft Drink, Monster, NESTEA *, NESTEA COOL *, Northern, Neck, NOS, Odwalla, Peace, Pepe Rico, Pibb, POWERADE, POWERADE LIGHT, POWERADE PLAY, Red Flash, Simply Orange, Smart, Sokenbicha, Southern Sun, Sprite, Sprite Remix, Sprite Zero/diet Sprite/Sprite light, Sunfill, TaB, Vanilla Coke, VAULT, Vegitabeta, Worx, Energy, Zico, all Pepsi brands and Gatorade brands.

Inositol phosphates and/or analogs described herein are excellent candidates for chemoprevention due to cell permeability, solubility, low toxicity, and demonstrated efficacies inhibiting tumor growth.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

### REFERENCES CITED IN THE ABOVE SPECIFICATION

Ishikawa T, Nakatsuru Y, Zarkovic M, Shamsuddin A M (1999) Inhibition of skin cancer by IP6 in vivo: initiation-promotion model. Anticancer Res 19:3749-3752.
Gupta K P, Singh J, Bharathi R (2003) Suppression of DMBA-induced mouse skin tumor development by inositol hexaphosphate and its mode of action. Nutr Cancer 46:66-72.
Kolappaswamy K, Williams K A, Benazzi C, Sarli G, McLeod C G Jr, Vucenik I, DeTolla L J (2009) Effect of inositol hexaphosphate on the development of UVB-induced skin tumors in SKH1 hairless mice. Comp Med 59:147-152.
Chen N, Ma W Y, Dong Z (2001) Inositol hexaphosphate inhibits ultraviolet B-induced signal transduction. Mol Carcinog 31:139-144.
Williams K A, Kolappaswamy K, Detolla L J, Vucenik I (2011) Protective effect of inositol hexaphosphate against UVB damage in HaCaT cells and skin carcinogenesis in SKH1 hairless mice. Comp Med 61:39-44.
El-Sherbiny Y M, Cox M C, Ismail Z A, Shamsuddin A M, Vucenik I (2001) G0/G1 arrest and S phase inhibition of human cancer cell lines by inositol hexaphosphate (IP6). Anticancer Res 21:2392-2403.
Estensen R D, Wattenberg L W (1993) Studies of chemopreventive effects of myo-inositol on benzo[a]pyrene-induced neoplasia of the lung and forestomach of female A/J mice. Carcinogenesis 14:1975-1977.
Gustafson A M, Soldi R, Anderlind C, Scholand M B, Qian J X, Zhang X, Cooper K, Walker D, McWillimas A, Liu G, Szabo E, Brody J, Massion P P, Lenburg M E, Lam S, Bild A H, Spira A (2010) Airway PI3K pathway activation is an early and reversible event in lung cancer development. 2:26ra25.
Gu M, Roy S, Raina K, Agarwal C, Agarwal R (2009) Inositol Hexaphosphate Suppresses Growth and Induces Apoptosis in Prostate Carcinoma Cells in Culture and Nude Mouse Xenograft: PI3K-Akt Pathway as Potential Target. Cancer Res 69:9465-9472.
Hecht S S, Upadhyaya P, Wang M, Bliss R L, McIntee E J, Kenney P M (2002) Inhibition of lung tumorigenesis in A/J mice by N-acetyl-S—(N-2-phenethylthiocarbamoyl)-L-cysteine and myo-inositol, individually and in combination. Carcinogenesis 23:1455-1461.
Huang C, Ma W Y, Hecht S S, Dong Z (1997) Inositol hexaphosphate inhibits cell transformation and activator protein 1 activation by targeting phosphatidylinositol-3′ kinase. Cancer Res 57:2873-2878.
Lam S, McWilliams A, LeRiche J, MacAulay C, Wattenberg L, Szabo E. (2006) A phase I study of myo-inositol for lung cancer chemoprevention. Cancer Epidemiol. Biomarkers Prev. 15:1526-1531.
Lee H J, Lee S A, Choi H (2005) Dietary administration of inositol and/or inositol-6-phosphate prevents chemically-induced rat hepatocarcinogenesis. Asian Pac J Cancer Prev 6:41-47.
Sakamoto K, Venkatraman G, Shamsuddin A M (1993) Growth inhibition and differentiation of HT-29 cells in vitro by inositol hexaphosphate (phytic acid). Carcinogenesis 14:1815-1819.
Schröterová L, Hasková P, Rudolf E, Cervinka M (2010) Effect of phytic acid and inositol on the proliferation and apoptosis of cells derived from colorectal carcinoma. Oncol Rep 23:787-793.
Shamsuddin A M, Elsayed A M, Ullah A (1988). Suppression of large intestinal cancer in F344 rats by inositol hexaphosphate. Carcinogenesis 9:577-580.
Shamsuddin A M, Uilab A, Chakravartby A K (1989) Inositol and inositol hexaphosphate suppress cell proliferation and tumor formation in CD-1 mice. Carcinogenesis 10(8): 1461-3.
Tian Y, Song Y (2006) Effects of inositol hexaphosphate on proliferation of HT-29 human colon carcinoma cell line. World J Gasroenterol 14:4137-4142.
Norazalina S, Norhaizan M E, Hairuszah I, Norashareena M S (2010) Anticarcinogenic efficacy of phytic acid extracted from rice bran on azoxymethane-induced colon carcinogenesis in rats. Exp Toxicol Pathol 62:259-268.
Ullah A, Shamsuddin A M (1990) Dose-dependent inhibition of large intestinal cancer by inositol hexaphosphate in F344 rats. Carcinogenesis 11:2219-22.
Witschi H, Espiritu I, Uveminami D (1999) Chemoprevention of tobacco smoke-induced lung tumors in A/J strain mice with dietary myo-inositol and dexamethasone. Carcinogenesis 20:1375-1378.

I claim:

1. An aqueous liquid formulation comprising:
(a) water and
(b) myoinositol hexaphosphate and/or an optical isomer thereof, and/or an orally acceptable salt thereof,
further optionally comprising one or more components selected from the group consisting of:
(1) myoinositol and/or an optical isomer;
(2) an orally acceptable free radical scavenger
(3) a nutritionally acceptable orally administrable electrolyte;
(4) a nutritionally acceptable orally administrable vitamin;
(5) a flavor;
(6) an orally administrable coloring agent;
(7) an orally administrable sweetener;
(8) an oral formulation acceptable thickener;
(9) an orally administrable, liquid formulation processing aid; and
(10) an orally administrable, liquid formulation auxiliary carrier other than water.

2. A method of reducing the risk of damage to cells or tissues due to reactive oxygen species free radicals in a subject in need thereof comprising orally administering the formulation of claim 1.

3. A method of preventing damage to cells or tissues due to reactive oxygen species free radicals in a subject in need thereof comprising orally administering the formulation of claim 1.

4. A method of treating damage to cells or tissues due to reactive oxygen species free radicals in a subject in need thereof comprising orally administering the formulation of claim 1.

5. A method of reducing the risk of developing a cancer selected from breast, pancreatic, ovarian, prostate, lung cancer, skin cancer, colon cancer liver, cervical, uterine, liver, blood, soft tissue, and muscular tumors in a subject in need thereof comprising orally administering the formulation of claim 1.

6. A method of preventing a cancer selected from breast, pancreatic, ovarian, prostate, lung cancer, skin cancer, colon cancer liver, cervical, uterine, liver, blood, soft tissue, and muscular tumors in a subject in need thereof comprising orally administering the formulation of claim 1.

7. A method of treating a cancer selected from breast, pancreatic, ovarian, prostate, lung cancer, skin cancer, colon cancer liver, cervical, uterine, liver, blood, soft tissue, and muscular tumors in a subject in need thereof comprising orally administering the formulation of claim 1.

8. A method of reducing adverse effects of diagnostic radiation treatments in a patient in need thereof comprising orally administering to such a patient, before, during, or after said diagnostic radiation treatment, the formulation of claim 1.

9. A method of reducing the risk of adverse effects of environmental radiation exposure to a human or animal in need thereof comprising administering to said human or said animal respectively, before, during, or after said environmental, radiation exposure, the composition of claim 1.

10. The method of claim 9 wherein said environmental radiation exposure is selected from the group consisting of:
a) extreme elevation above sea level;
b) flying;
c) going into planetary orbit or further into space; and
d) mining, purification, or handling of radioactive materials.

11. A method of reducing the risk of adverse effects of environmental exposure to at least one of air pollutants, cigarette smoke, and/or other free oxygen radical generating substances in a human or animal comprising administering to said human or said animal respectively, before, during, or after said exposure, the composition of claim 1.

12. The product of claim 1 which is a liquid nutritional supplement.

13. The product of claim 1 which is an orally administered product.
